(19) United States
(12) Patent Application Publication
(10) Pub. No.: US 2014/0088508 A1
(43) Pub. Date: Mar. 27, 2014

(54) DRUG-DELIVERY DEVICES WITH INTEGRATED NEEDLE-INSERTION MECHANISM

(71) Applicants: Patrick Ryan, Los Angeles, CA (US); Po-Ying Li, Monrovia, CA (US); Conrad Diaz, Pasadena, CA (US); Brett Daniel Schleicher, New York, NY (US); Sean Caffey, Hawthorne, CA (US)

(72) Inventors: Patrick Ryan, Los Angeles, CA (US); Po-Ying Li, Monrovia, CA (US); Conrad Diaz, Pasadena, CA (US); Brett Daniel Schleicher, New York, NY (US); Sean Caffey, Hawthorne, CA (US)

(21) Appl. No.: 14/035,848

(22) Filed: Sep. 24, 2013

Related U.S. Application Data
(60) Provisional application No. 61/704,974, filed on Sep. 24, 2012.
(60) Provisional application No. 61/875,470, filed on Sep. 9, 2013.

(51) Int. Cl. A61M 5/158

(52) U.S. Cl. CPC A61M 5/158
USPC 604/152

(57) ABSTRACT

Drug-delivery devices for subcutaneous injections may be provided with pre-filled drug reservoirs and/or integrated needle-insertion mechanisms to increase ease of use of the devices. Certain device configurations include a needle disposed above or below the drug vial, parallel to the bottom wall of the device, with the needle-insertion mechanism placed at least partially side-by side with the vial.

### CROSS-REFERENCE TO RELATED APPLICATION

The present application claims priority to and the benefit of, and incorporates herein by reference in their entireties, U.S. Provisional Applications No. 61/704,974, filed on Sep. 24, 2012, and No. 61/875,470, filed on Sep. 9, 2013.

### TECHNICAL FIELD

The present invention relates to various aspects of drug pump devices and their operation, in particular, in various embodiments, to electrolysis pumps and associated control methods as well as to needle-insertion systems for subcutaneous drug infusions.

### BACKGROUND

Subcutaneous drug delivery is the delivery of drugs to the subcutaneous tissue (i.e., the layer below the skin), and is a very common route of drug administration. In cases where slow absorption of a drug (such as, e.g., insulin) is required, subcutaneous delivery may even be the only allowable drug-administration method. Advantages of subcutaneous delivery over other routes of administration, such as intravenous delivery, include its suitability for at-home injections, increased convenience, and the resulting improvement in the patient's quality of life.

Conventionally, subcutaneous injections have been performed manually with syringes or pre-filled pen injectors via a needle inserted into the patient. Manual drug injection into subcutaneous tissue can, however, cause discomfort or even severe pain to the patient if drug volumes larger than 1 mL are injected, and may leave undesired trauma at the injection site. Furthermore, it is generally difficult for the patient or medical staff to hold a syringe plunger for a longer time to administer a large dosage. Therefore, drug volumes delivered subcutaneously through a single syringe injection are typically kept below 1 mL or, at most, 1.2 mL. This is a severe limitation for protein-based drug therapies (including, e.g., monoclonal antibody therapies), which frequently require larger volumes to be delivered, requiring patients to undergo multiple injections during a single dosing cycle to receive the appropriate amount. To obviate the need for large-volume injections, drug companies have spent a significant amount of time and money to make more concentrated formulations. This is, however, difficult and costly, and introduces other issues, such as increased viscosity.

To accommodate the need for larger dosages and/or subcutaneous drug delivery over extended periods of time, various automatically operated, portable drug-delivery devices with passive or electronically controlled active pump mechanisms (e.g., motor-driven or electrolysis pumps) have been developed. For example, many insulin-infusion pumps for diabetes treatment are on the market. Unfortunately, conventional insulin (or other drug) pumps are sold without the drug, requiring the user to fill the drug reservoir prior to deployment; this process is complex and time-consuming, and introduces the potential for user error (such as selection of the wrong drug or amount, or the introduction of air into the drug reservoir). Further complicating self-administration of the drug, the needle and/or cannula used to inject the drug into the subcutaneous tissue are often provided separately from the device (requiring a fluidic connection between the drug reservoir and the needle or cannula to be manually established), as is the insertion mechanism used to push the needle/cannula through the skin. To increase ease of use and safety for the patient, it is highly desirable to provide devices and methods that obviate the need for manual drug-filling and/or needle-insertion.

### SUMMARY

The present invention provides drug-delivery devices with pre-filled drug reservoirs and integrated needle/cannula insertion mechanism. In various embodiments, the drug-delivery device is assembled from a pre-filled, standard drug vial, a plunger fitted into the vial, and a pump (e.g., an electrolysis pump) placed at the back end of the vial behind the plunger; the pump causes the plunger to move towards an outlet of the vial to expel liquid drug therefrom. The vial outlet is fluidically connected (e.g., via soft tubing, which may include a vent or filter allowing air to pass) to the needle and/or cannula intended for insertion into the patient's subcutaneous tissue.

In some embodiments, the vial is oriented parallel to the bottom wall of the device housing, which is, during use, placed against the user's skin. The needle and/or cannula may be disposed above or below, and parallel to, the vial. A curved needle guide may be used to bend the needle near its tip so as to guide it at an angle towards an opening in the bottom wall. In implementations where the needle passes under the vial, the needle guide may be curved to guide the needle first away from the bottom wall and then back toward the opening in the bottom wall, thereby increasing the bend radius to prevent damage to the needle. The insertion mechanism may include needle and/or cannula carriers that are propelled forward by a compressed insertion spring upon activation of the mechanism. A retraction spring may also be included to allow retraction of the needle only following insertion of the needle and cannula under the skin. In some embodiments, the insertion and/or retraction springs are placed off-axis from the needle and cannula, side-by-side with the vial, reducing the profile of the drug-delivery device.

Accordingly, in one aspect, the invention is directed to a drug pump device including a housing with a bottom wall having an outer surface for placement in contact with a patient's skin and, contained within the housing, a drug vial oriented parallel to the bottom surface and having an outlet at a first end thereof and forming a drug reservoir therein, a plunger movably disposed within the vial, a pump attached to the drug vial at a second end thereof for causing movement of the plunger so as to expel drug from the outlet of the vial, a needle fluidically connectable to the outlet of the drug vial and disposed either above or below the vial and parallel thereto, an insertion mechanism for advancing the needle along an axis thereof, a curved needle guide, disposed on the bottom wall and aligned with the axis of the needle at an upper end, for bending the needle upon advancement thereof so as to guide a tip of the needle toward and through the bottom wall and/or a plane coincident therewith (i.e., a plane containing the bottom wall). The bottom wall may include an opening, and the needle guide may be aligned, at a lower end thereof, with the opening so as to guide the needle tip therethrough. In some embodiments, the needle is disposed below the vial and the needle guide is curved to guide the needle first away from the bottom wall and then back toward the opening in the bottom wall. Further, the device may include a skin-adhesive patch attached to the outer surface of the bottom wall. In some embodiments, the device includes a fluid-path connector for connecting the outlet of the drug vial to the needle; the fluid-path connector may have a vent permeable to air but not liquid.

The insertion mechanism may include a needle carrier slidably movable relative to the housing in a direction parallel to the needle axis, and optionally further an insertion spring for advancing the needle so as to advance the needle through the bottom wall of the housing (or a plane therethrough). The insertion spring may be placed off-axis from the needle and side-by-side with the vial. The insertion mechanism may further include a retraction spring for retracting the needle carrier whereby the needle is retracted into the housing; the retraction spring may likewise be placed off-axis from the needle, and concentrically with the insertion spring.

The device may further include a cannula disposed concentrically with the needle and fluidically connectable thereto. The needle may ride within the cannula or, alternatively, the needle may be hollow and the cannula may ride therein. The insertion mechanism may further include a cannula carrier slidably movable relative to the housing in a direction parallel to the cannula axis. In some embodiments, the insertion mechanism, moreover, includes a latch-and-release structure for causing the needle and cannula carriers to move together during advancement and, upon insertion of the needle and cannula through the bottom surface of the housing, detaching the cannula carrier from the needle carrier to allow movement of the needle carrier relative to the cannula carrier. The device may also include a manually operable trigger mechanism for triggering the insertion mechanism.

In another aspect, the invention pertains to a drug pump device including a housing and, contained therein, a drug vial having an outlet at a first end thereof and forming a drug reservoir therein, a plunger movably disposed within the vial, a pump attached to the drug vial at a second end thereof for causing movement of the plunger so as to expel drug from the outlet of the vial (the vial being oriented parallel to the bottom surface), a needle fluidically connectable to the outlet of the drug vial, and a fluid-path connector for connecting the outlet of the drug vial to the needle, the fluid-path connector comprising a vent permeable to air but not liquid.

In yet another aspect, the invention relates to a drug pump device including a housing comprising a bottom wall having an outer surface for placement in contact with a patient's skin and, contained within the housing, a drug vial having an outlet at a first end thereof and forming a drug reservoir therein, a plunger movably disposed within the vial, a pump attached to the drug vial at a second end thereof for causing movement of the plunger so as to expel drug from the outlet of the vial (the vial being oriented parallel to the bottom surface), a needle fluidically connectable to the outlet of the drug vial and disposed below the vial and parallel thereto, and a curved needle guide, disposed on the bottom wall and, aligned, at an upper end thereof, with the axis of the needle, for bending the needle upon advancement thereof so as to guide a tip of the needle first away from the bottom wall and then back toward an opening in the bottom wall.

### BRIEF DESCRIPTION OF THE DRAWINGS

The foregoing will be more readily understood from the following detailed description, in particular, when taken in conjunction with the drawings, in which:

FIG. 1A is a schematic cross-sectional view of an electrolytically driven drug pump device in accordance with various embodiments;

FIG. 1B is a top view of an interdigitated electrode configuration in accordance with various embodiments;

FIGS. 2A-2C are cross-sectional views of lifetime-extending electrode/substrate configurations in accordance with various embodiments;

FIG. 3 is a block diagram schematically illustrating a control system for drug pump devices in accordance with various embodiments;

FIG. 4A is a graph illustrating the pump-chamber pressure measured as a function of time for an exemplary drug-delivery device in accordance with various embodiments;

FIG. 4B is a graph illustrating the delivered drug volume computed from the pump-chamber pressure of FIG. 4A in accordance with various embodiments;

FIG. 4C is a graph illustrating the flow rate derived from the delivered drug volume of FIG. 4B in comparison with a directly measured flow rate in accordance with various embodiments;

FIG. 4D is a graph illustrating an end-of-dose pressure signature in accordance with various embodiments;

FIG. 5A is a perspective view of a drug-delivery device with an integrated insertion system in accordance with various embodiments;

FIG. 5B is a perspective view of the housing of the drug-delivery device of FIG. 5A;

FIG. 5C is a schematic close-up view of a fluid-path connection between the drug vial of a drug-delivery device as depicted, e.g., in FIG. 5A and the injection site in accordance with various embodiments;

FIGS. 6A-6C are schematic close-up views of different needle guides in accordance with various embodiments;

FIGS. 7A and 7B are top and side views, respectively, of the drug-delivery device of FIG. 5A prior to actuation in accordance with various embodiments;

FIG. 7C is a close-up view of a latch mechanism used in the drug-delivery device of FIG. 5A in accordance with various embodiments; and

FIGS. 7D and 7E are top and side views, respectively, of the drug-delivery device of FIG. 5A following activation and needle insertion in accordance with various embodiments.

### DETAILED DESCRIPTION

### Electrolysis Pump Devices and Pump-Control Methods

Various embodiments of the present invention provide portable electrolysis-driven drug pump devices capable of delivering in excess of 1 mL (preferably 3 mL or more) of drug without refilling the drug reservoir. Such devices are particularly useful for drug therapies that require large dose volumes, such as many biologics (i.e., drugs created by biological processes rather than chemically), including, for instance, monoclonal antibodies, insulin, or other protein-based drugs. Advantageously, portable electrolysis pump devices facilitate accurately dosed automatic injections over longer periods of time (as compared with traditional syringe injections), which reduces pain and discomfort and increases the convenience of drug administration. In some embodiments, the drug pump devices are supplied with pre-filled drug containers, e.g., in the form of standard glass or polymer drug vials or cartridges, eliminating the need for the patient to fill the reservoir prior to use; typically, such pre-filled pump devices are designed for one-time use and subsequent disposal.

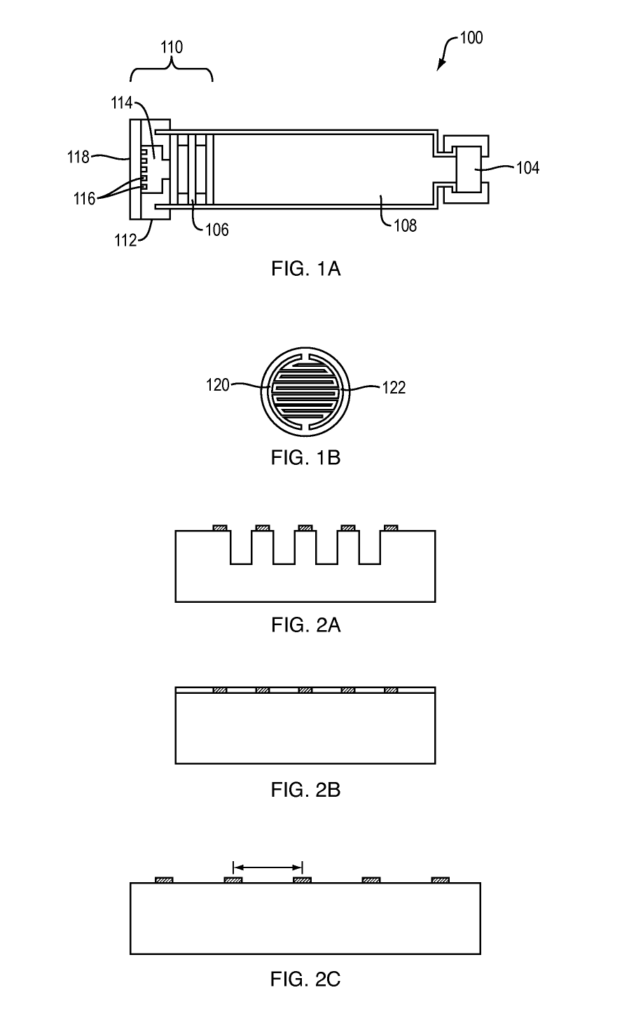

FIG. 1A schematically illustrates an exemplary drug pump device 100 in accordance herewith. The device 100 includes a drug vial or cartridge 100, which is closed, at the outlet end, by a pierceable, self-sealing septum 104. A plunger 106 is slidably disposed within the vial 102, forming a drug reservoir 108 in the vial 102 between the plunger 106 and the septum 104. During deployment of the device for drug delivery, a needle may pierce the septum 104 to establish fluid communication between the reservoir 108 and a downstream fluid path. Behind the plunger 106, an electrolysis pump 110 is attached and sealed to the back end of the vial 102. The pump 110 may, for instance, include a housing 112 that is, at an open end, inserted into or placed over the back end of the vial 102 or, as shown, includes a grove for receiving a portion of the vial wall therein. An O-ring, gasket, or other seal (not shown) may be placed between the contacting surfaces of the vial 102 and pump housing 112, and a clamping force may be applied simultaneously from both the front of the vial 102 and the back of the pump housing 112 (e.g., using a screw, snap-in mechanism, or clamp) to tightly seal the device.

The pump housing 112 forms a pump chamber 114, which contains one or more pairs of electrodes 116 and, in contact therewith, an electrolyte. In some embodiments, the electrolyte is provided in liquid form. In other embodiments, the electrolyte is absorbed within a matrix (e.g., a hydrogel, cotton ball, or other absorbent material) placed within the pump chamber adjacent or surrounding the electrodes; the matrix may serve to ensure contact between the electrodes and the electrolyte regardless of the device orientation (see, e.g., U.S. patent application Ser. No. 13/091,047, entitled “Electrolytically Driven Drug Pump Devices”, filed on Apr. 20, 2011, which is hereby incorporated herein by reference in its entirety). The electrodes 116 may be, e.g., wires, pins, foil segments, wire loops, and/or microelectrodes, and may extend far into interior of the pump chamber 114 or, as shown, be disposed on a flat substrate 118, which may also form the back wall of the pump housing 112.

During pump operation, the electrodes 116 are driven by a battery or other power source (not shown) to break the electrolyte into gaseous products. Suitable electrolytes include water and aqueous solutions of salts, acids, or alkali, as well as non-aqueous ionic solutions. The electrolysis of water, for example, involves the following chemical reactions:

$$anode \text{:} 2 {H}_{2} O ( l ) {{⇄}_{recombination}}^{electrolysis} {O}_{2} ( g ) + 4 {H}^{+} ( aq ) + 4 {e}^{-}$$

$$cathode \text{:} 2 {H}_{2} O ( l ) + 2 {e}^{-} {{⇄}_{recombination}}^{electrolysis} 2 {OH}^{-} ( aq ) + {H}_{2} ( g )$$

$$Net \text{:} 2 {H}_{2} O ( l ) {{⇄}_{recombination}}^{electrolysis} {O}_{2} ( g ) + 2 {H}_{2} ( g )$$

The net result of these reactions is the production of oxygen and hydrogen gas, which causes an overall volume expansion of the pump chamber 114, pushing the plunger 106 forward and thereby expelling drug from the reservoir 108. As an alternative (or in addition) to water, ethanol may be used as an electrolyte, resulting in the evolution of carbon dioxide and hydrogen gas. Ethanol electrolysis is advantageous due to its greater efficiency and, consequently, lower power consumption, compared with water electrolysis. Regardless of the particular electrolyte used, electrolysis provides a simple and efficient means of generating pump pressure, and can be implemented in a small, low-cost pump. For example, in some embodiments, a compact micro-electrolysis pump with an initial pump-chamber volume of only about 100 μL (or less) can deliver the entire contents of a 3-mL standard drug vial. The pump may be capable of providing a large driving force (up to 200 psi) with low power consumption (on the order of a few mW) and a low drive voltage (in the range from 3 to 5 V).

In various embodiments, the electrolysis pump uses microelectrodes disposed on a flat substrate. FIG. 1B shows, for example, an arrangement of interdigitated cathode and anode structures 120, 122; along direction 124 (which corresponds to the cross-sectional view of FIG. 1A), the prongs 126 of the cathode and anode 120, 122 are arranged in an alternating fashion. Such interdigitation generally provides for good electrode performance—i.e., low drive-voltage requirements for a given desired drive current and, thus, low power consumption—as the overall surface area of the electrodes 120, 122 is large. In another embodiment, multiple electrode pairs are used to increase the pump efficiency and lower the power consumption; a multi-electrode configuration can also provide redundancy and increase the safety and reliability of the pump. Each electrode pair may be connected to its own battery, conserving battery capacity (since multiple individual batteries are less quickly depleted during pump operation periods than a single battery that drives all electrode pairs) and, thus, prolonging battery lifetime (of course, at the cost of needing multiple batteries and increasing the device size). Alternatively, all electrode pairs may be connected in parallel to the same battery (or other power source); each electrode pair then generates electrolysis gas at a fraction of 1/n compared with n individually powered electrode pairs. If multiple electrode pairs are used, their cathodes and anodes may be arranged alternatingly, resulting in a similar cross-sectional structure as the interdigitated electrodes shown in FIG. 1B.

Microelectrodes in accordance herewith may be manufactured, e.g., by micromachining, printed-circuit-board techniques, screen printing, electrical-discharge machining (EDM), computer-numerical-control (CNC) machining, or electroplating. Among these techniques, all of which are well-known to persons of skill in the art, PCB methods allow for low-cost electrode manufacturing, and micromachining provides good electrode performance (i.e., high gas-generation efficiency) and mechanical stability. A performance and stability comparable to that of micromachined microelectrodes can also be achieved by electroplating electrodes manufactured on a printed circuit board (PCB). PCBs typically use copper or aluminum for the electronic circuitry; these materials generally corrode quickly during electrolysis and, consequently, tend to peel off or dissolve. Electroplating a chemically more stable material onto the copper or aluminum metal core may prevent corrosion and prolong electrode life (while retaining the lower cost of PCB manufacturing as compared to, e.g., microelectromechanical-systems-(MEMS)-based manufacturing processes). Many plating metals can be used for this purpose, including, e.g., platinum, palladium, iridium, gold, silver, other noble materials, or certain alloys. The particular metal used may depend on a number of pump conditions and pump-performance criteria, including, e.g., the desired gas-generation rate, the electrolyte used, any limits on acceptable voltage and/or current requirements (as imposed, e.g., by size constraints for the power source), the required pump (and, thus, electrode) lifetime, and the requisite structural integrity of the electrode structure to achieve a desired uniformity and/or controllability of electrolysis. Iridium, for example, facilitates electrolysis at low drive voltages, and palladium, platinum, and (to a lesser extent) gold offer high long-term stability due to slow oxidation rates.

High electrode stability is particularly important for high-flow-rate applications (e.g., drug injection at rates of a few hundred μL/min to a few mL/min), since the high electrical drive currents generally required to sustain the high flow rates tend to enhance mechanisms that negatively affect the structural integrity and performance of the microelectrodes; these mechanisms include, in particular, metal delamination, metal oxidation, metal re-deposition, and metal dissolution. The risk for delamination can be reduced by selecting a metal that ensures good adhesion between the bottom seed layer and the top reactive layer. Platinum, for instance, adheres better to gold than copper or aluminum adhere to gold. To combat metal oxidation, re-deposition, and dissolution, an inert metal such as gold, palladium, iridium, and platinum may be used. Platinum is the preferred choice since it provides the most inert surface for electrochemical reactions and is characterized by the lowest oxidation and dissolution rates among metals suitable as electrode materials. There are many plating solutions that can be used for electroplating platinum onto electrodes: dinitroplatinite sulfate, chloroplatinic acid, P salt [(NH₃)₂Pt(NO₂)₂], and Q salt [(NH₃)₄Pt(HPO₄)], to name just a few. P-salt-plated platinum (which is widely used in the aircraft industry for engine-blade coating) provides electrodes with particularly good performance and stability.

However, if platinum is not available (e.g., because its use is cost-prohibitive) and oxidation and re-deposition can be expected occur, other techniques may be used to extend the lifetime of the electrodes. In one approach, shown in FIG. 2A, the substrate material is etched between the opposing electrodes (e.g., as shown, between the alternating prongs of interdigitated electrodes or between alternating cathodes and anodes of a multi-electrode-pair arrangement) to form a trench preventing re-deposition. A deep trench can also be pre-formed prior to electrode deposition. The particular method to create the trenches may be selected based on the substrate material. For example, for silicon substrates, wet-etching with potassium hydroxide (KOH) may be suitable; glass may be wet-etched with hydrogen fluoride (HF), and plastic may be wet-etched with acids (such as sulfuric acid (H₂SO₄)) or bases (such as KOH). Silicon or plastic substrates (e.g., of polyether ether ketone (PEEK), polyethylene (PE), or para-methoxymethamphetamine (PMMA)) may, alternatively, be dry-etched by plasma etching, reactive-ion etching (RIE), or deep reactive-ion etching (DRIE). In glass substrates, trenches may be created by computer-numerical-control (CNC) milling. All of these techniques are well-known to persons of skill in the art.

In another approach, illustrated in FIG. 2B, a mask material, such as, generally, any electrically insulating material, is deposited between the electrodes to retard re-deposition. This method is similar to the solder-masking process that is widely used in the PCB industry for solder/metal protection. The masking material is usually patternable. Example of suitable materials include screen-printable materials such as epoxy liquid, liquid photoimageable solder mask (LPSM) inks, and dry-film photoimageable solder mask (DFSM). Other possible materials include UV-photopatternable materials such as epoxy (SU-8), PMMA, liquid photoresist, and dry-film photoresist. Methods for applying these fluids include, e.g., spin-coating, spray-on, and screen-printing. For spin-coating and spray-on methods, standard photolithography is employed, involving the definition and removal of material from the electrode area, leaving the masking material between the electrodes. For the screen-printing method, ink fluid is applied, squeegeed using a screen, and dried to create the masking pattern. The dry film may be peeled off the backing and applied to the PCB, and then photolithography patterned.

In a third approach, shown in FIG. 2C, the electrode structure includes, by design, a wide distance between anode and cathode structures, lengthening the re-disposition route. The distance between electrodes may, for example, be in the range from tens of micrometers to hundreds of micrometers. Typically, the distance is selected based on the electrolyte composition, the current required for the application, the intended product life, and/or the electrolysis cycles employed, etc. to strike a balance (i.e., optimize the trade-off) between electrolysis efficiency, cost, and minimized lifetime failures.

In electrolysis pumps, pump pressure can be straightforwardly controlled via the drive current supplied to the electrolysis electrodes: the amount of gas generated is proportional to the drive current integrated over time, and can be calculated using Faraday's law of electrolysis. For example, creating two hydrogen molecules and one oxygen molecule from water requires four electrons; thus, the amount (measured in moles) of gas generated by electrolysis of water equals the total electrical charge (i.e., current times time), multiplied by a factor of ¾ (because three molecules are generated per four electrons), divided by Faraday's constant. As more gas is generated in the pump chamber, the volume of the chamber, the pressure in the chamber, or—generally—both increase; typically, the gas within the chamber can be treated, in good approximation, with the ideal-gas law, and the product of pump-chamber volume and pressure is, thus, proportional to the amount of gas (measured in moles). Any volume increase of the chamber translates directly into an equal volume of drug expelled from the drug reservoir. Accordingly, the drug flow rate equals the time derivative of the chamber volume.

The ratio of pump pressure to flow rate is given by the flow resistance of the downstream fluid path and any back pressure at the injection site. The flow resistance, in turn, is generally a function primarily of the fluid-path dimensions (e.g., the inner diameter of a needle or cannula downstream the reservoir), but can be affected significantly by occlusions in that path as well as by any friction between vial and plunger. Thus, while the flow resistance can, in principle, be derived from the device specifications and/or calibrated, it is generally desirable to also monitor the pump pressure and/or the flow rate during drug delivery. The measured parameters may then be fed into a feedback loop to control pump operation to achieve a desired flow rate and/or a desired dosage.

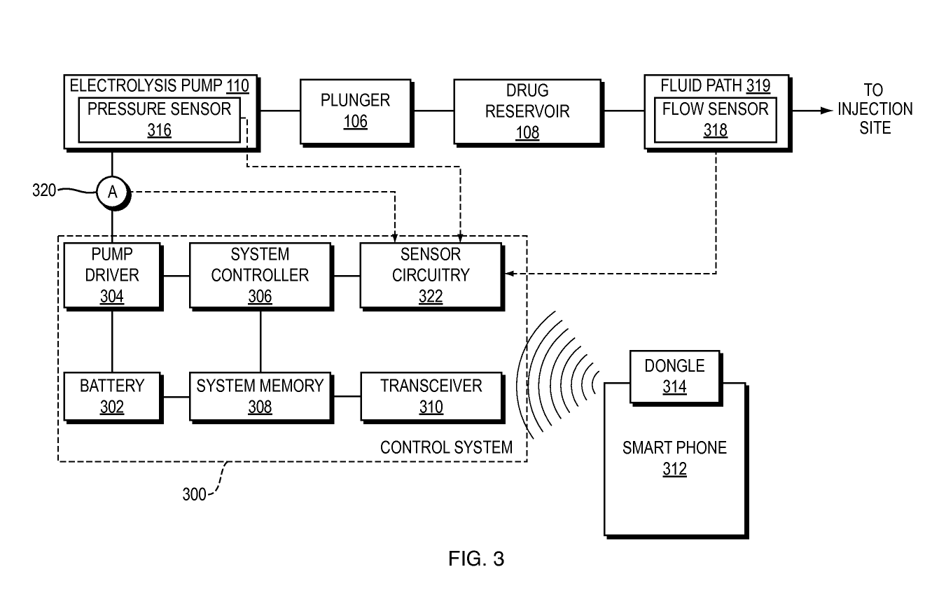

FIG. 3 conceptually illustrates the components a drug-pump control system 300 and its interrelation with the other pump device components in accordance with various embodiments. The control system 300 includes a battery 302 (e.g., a non-rechargeable lithium battery, or a rechargeable lithium-ion, lithium polymer, nickel-metal-hydride, or nickel-cadmium battery) or other power source (such as a capacitor, solar cell, or motion-generated energy system) and the electronic circuitry necessary for driving the electrolysis pump 110. More specifically, the electronic circuitry may include a pump driver 304 that sets the drive current based on signals from a system controller 306 (e.g., a microcontroller). The system controller 306 may be a microcontroller, i.e., an integrated circuit including a processor core, memory (e.g., in the form of flash memory, read-only memory (ROM), and/or random-access memory (RAM)), and input/output ports. The memory may store firmware that directs operation of the drug pump device. In addition, the device may include read-write system memory 308. In certain alternative embodiments, the system controller 306 is a general-purpose microprocessor that communicates with the system memory 308.

The system memory 308 (or memory that is part of a microcontroller) may store a drug-delivery protocol (specifying, e.g., drug delivery times, durations, rates, and dosages) in the form of instructions executable by the controller 306, which may be loaded into the memory at the time of manufacturing, or at a later time by data transfer from a hard drive, flash drive, or other storage device, e.g., via a USB, Ethernet, or firewire port. In alternative embodiments, the system controller 306 comprises analog circuitry designed to perform the intended function (e.g., to deliver the entire bolus upon manual activation by the patient). In certain embodiments, the control system 300 include a signal receiver (for uni-directional telemetry) or a transmitter/receiver 310 (for bi-directional telemetry) that allows the device to be controlled and/or re-programmed remotely by a wireless handheld device, such as a customized personal digital assistant (PDA) or a smartphone 312. The smartphone 312 may communicate with the control system 300 using a connection already built into the phone, such as a Wi-Fi, Bluetooth, or near-field communication (NFC) connection. Alternatively, a smartphone dongle 314 (i.e., a special hardware component, typically equipped with a microcontroller, designed to mate with a corresponding connector on the smartphone) may be used to customize the data-transfer protocol between the smartphone and the control system 300.

The control system 300 may be responsive to sensor measurements from one or more sensors embedded in various components of the drug pump device, such as a pressure sensor 316 in the pump chamber, one or more flow sensors 318 and/or pressure sensors in the drug reservoir and/or the cannula or other fluid-path component 319 downstream of the reservoir, and/or an Ampere-meter 320 measuring the current through the electrodes. Accurate micro-flow sensors and micro-pressure sensors suitable for use in small, portable pump devices are commercially available. With present micro-technology, micro-flow sensors generally cost much more than micro-pressure sensors, rendering control methods that rely on pressure-sensor measurements for monitoring pressure, volume, and flow rate an attractive low-cost solution.

The sensor feedback may be processed by dedicated sensor circuitry 322 and/or by the system controller 306 and used in conjunction with the pre-programmed drug-delivery protocol and/or real-time control input to monitor drug delivery and control pump operation based thereon; various suitable control mechanisms are described in detail in U.S. patent application Ser. No. 13/680,828 (entitled “Accurate Flow Control in Drug Pump Devices”), filed on Nov. 19, 2012, which is incorporated herein by reference in its entirety. When rapid infusion is the main goal and flow accuracy is less important, a simple pressure-based feedback loop may be used as a safety mechanism to monitor the drug pump device for over-delivery, under-delivery, occlusions within the fluid path, and/or the end of pumping (which occurs when the plunger 106 reaches the end of the vial). For many applications involving the dispensing of liquid, however, it is desirable to monitor and/or accurately control the flow rate (e.g., within the range from μL/min down to pL/min) and/or the cumulative volume of drug dispensed during a time period. In some embodiments, this is achieved by directly measuring the flow rate using a flow sensor downstream of the drug reservoir, and calculating the volume from the flow rate. Flow sensors, however, are not only expensive, but generally ought to be made of a biocompatible material as they come into contact with the drug. Therefore, in advantageous alternative embodiments, the delivered volume is calculated indirectly using a low-cost pressure sensor upstream of the reservoir in the electrolysis chamber of the device, where the sensor does not come in contact with the drug.

The flow rate and cumulative volumes delivered by an electrolysis pump device can be determined based on continuous measurements of the pressure in the electrolysis pump chamber and the electrolysis current supplied to the electrodes, under the following assumptions: (1) all gases in the pump chamber, including air, hydrogen, and oxygen produced by electrolysis, are ideal gases; (2) the environment sustains constant temperature and atmospheric pressure (i.e., 14.7 psia) throughout drug delivery; (3) there is no leakage of gas from the electrolysis chamber (or any non-zero leakage is negligibly small); and (4) all relevant device components, including the drug vial, pump seal (e.g., O-ring), and electrolysis chamber are rigid, i.e., do not exhibit any compliance (or change in volume) throughout delivery. If these assumptions are satisfied to a good approximation, the flow rate and/or delivered volume can be computed with significant accuracy. Even if an assumption does not hold, calibrations can typically compensate for any deviation therefrom (allowing the assumptions to be considered valid for purposes of the computations).

From the measured electrolysis current, the rate of electrolysis-gas generation can be derived as explained above; integrating the gas-generation rate over time yields the amount of electrolysis gas generated (in moles). Using the ideal gas law,

p·V=n·R·T,

the computed amount nₑₗ of electrolysis gas can be converted into the volume Vₑₗ occupied by the gas for any given pressure p (and temperature T). Thus, based on measurements of the pressure p in the pump chamber, the volume Vₑₗ of electrolysis gas in the chamber may be determined. A change in the volume of the pump chamber corresponds to a volume of liquid drug delivered from the reservoir. In practice, however, the pump-chamber volume changes not only as a result of electrolysis-gas generation, but also due to the compression of any air that is in the pump chamber even prior to pump operation. The “dead volume” V₀ occupied by the air initially, i.e., before electrolysis begins, is generally known from pump design (and is typically small compared with the volume of the drug vial). Assuming that the pump chamber is initially under atmospheric pressure pₐₜ, the contribution Vₐᵢᵣ of the air to the overall volume of the pump chamber is, at a later time for a pressure p in the pump chamber:

Vₐᵢᵣ=V₀·pₐₜ/p.

Thus, the pump-chamber volume for pressure p can be computed as:

V=1/p·(V₀·pₐₜ+nₑₗ·R·T).

The cumulative delivered drug volume may then be computed as the difference between the current pump-chamber volume V and the dead volume V₀, and the flow rate may be derived from the delivered volume by differentiation. In some embodiments, the electrolysis pump chamber is evacuated prior to deployment of the device, dispensing with the need to account for the dead volume.

In some embodiments, the electrolysis-gas volume is not computed based on theoretical considerations, but is determined empirically. For example, in an open-benchtop test setup, the electrolysis pump may be operated under atmospheric pressure Pₐₜ (or some other known, constant pressure p₁) and the gas-generation Q_{gg} rate be measured as a function of drive current I. In one test setup, the gas-generation rate was determined to be about Q_{gg}=(12/(1A)·I−23.7)(μL/min). From the experimentally determined gas-generation rate Q_{gg}, the cumulative volume Vₑₗ₁ of electrolysis gas may then be computed by integrating over time, or, for a constant drive current, by multiplying the gas-generation rate with the pumping time:

Vₑₗ₁=Q_{gg·t},

For a given volume V₁ of gas at pressure p₁, the corresponding volume V₂ of the same amount (in moles) of gas at pressure p₂ can be calculated using Boyle's law (which is derived from the ideal gas law):

p₁V₁=p₂V₂.

Taking the dead volume V₀ of air in the electrolysis chamber into consideration, the pump-chamber volume at pressure p can, thus, be computed as:

V=pₐₜ/p·(V₀+Q_{gg}·t)

As with the above-described approach using a computationally determined electrolysis gas volume, the cumulative delivered drug volume can be computed as the difference between the current pump-chamber volume V and the dead volume V₀, and the flow rate may be derived from the delivered volume by differentiation.

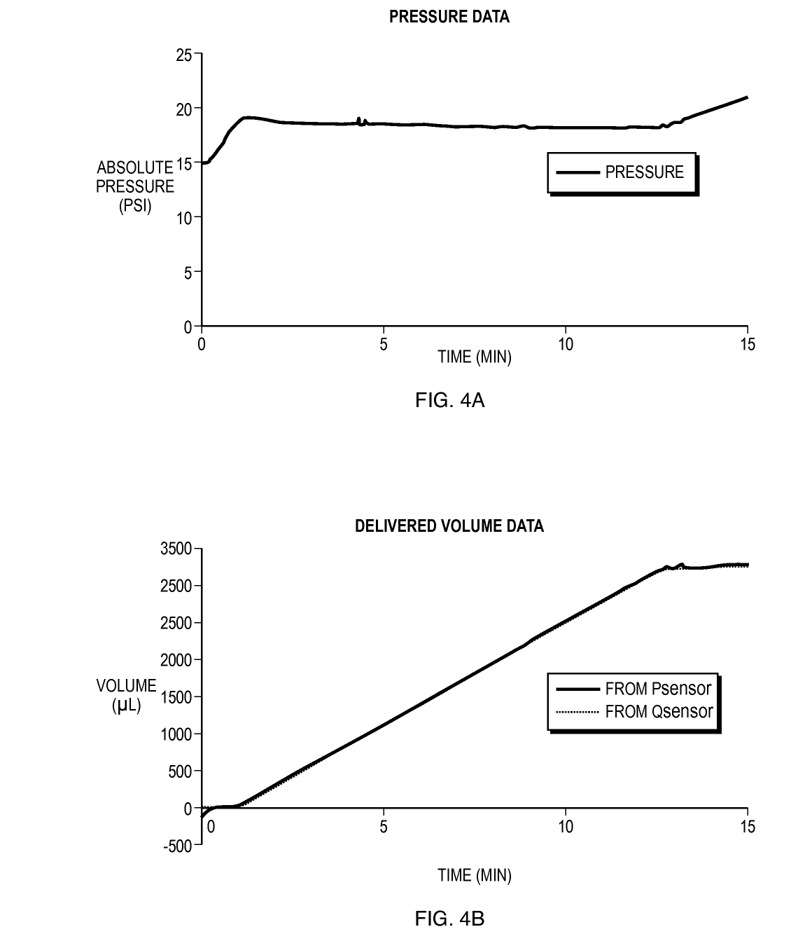

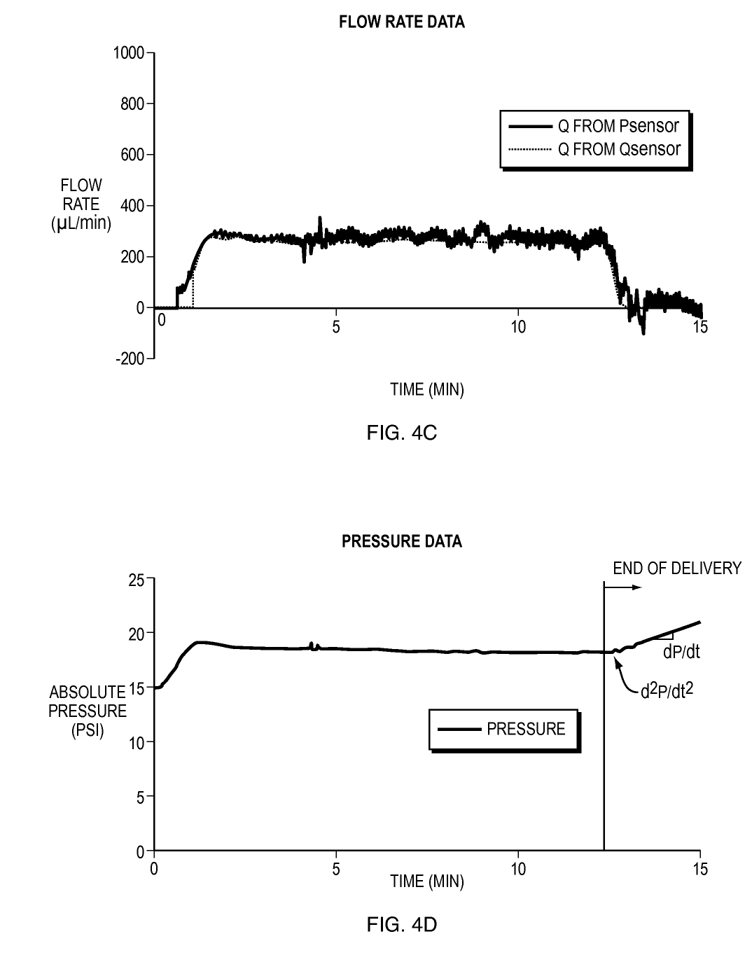

FIGS. 4A-4C illustrate the feasibility of flow-rate determinations via pressure measurements in the pump chamber as described above. FIG. 4A shows pump-pressure data as a function of time, and FIG. 4B shows the delivery volume derived from the measured pump pressure. FIG. 4C shows the delivery rate computed, by differentiation, from the delivery volume depicted in FIG. 4B, in comparison with the delivery rate as measured directly with a flow sensor. “Psensor” denotes data from an absolute-pressure sensor in the pump chamber, and “Qsensor” denotes a direct measurement or calculation from a downstream flow-sensor signal; as can be seen, the agreement between computed and directly measured flow rates is within the fluctuations of the measured signal.

Yet another way of determining the delivered drug volume and flow rate involves monitoring (and integrating over) incremental changes in the pump pressure and the amount of electrolysis gas. The incremental increase dV in the volume of the gas chamber may be computed, based on the ideal gas law, directly from the incremental changes in the electrolysis gas amount (dnₑₗ) and the pump-chamber pressure (dp):

dV=−(nₑₗ+n₀)·R·T/p²·dp+R·T/p·dnₑₗ.

Herein, n₀ is the amount of air (in moles) initially in the chamber (corresponding to the dead volume V₀); after the electrolysis pump has operated for a while, this amount may become negligibly small. Note that, unlike the amount of electrolysis gas nₑₗ, the amount of air does not change. From the above equation, it follows that the flow rate can be determined based on the rate of change in the pump pressure and the electrolysis gas generation rate dn/dt (both of which can be derived straightforwardly from continuously acquired pressure and electrolysis-current data) according to:

dV/dt=−n·R·T/p²·dp/dt+R·T/p·dn/dt.

In practice, the pressure-sensor signal contains noise that, without further processing, affects the delivery-volume computations according to any of the methods described above. Therefore, in various embodiments, a real-time filter, such as, e.g., a Kalman filter, moving-average filter, running-average filter, or band-pass filter is used to eliminate or at least decrease the noise (thereby increasing the signal-to-noise ratio (SNR)); these filters are well-known to persons of skill in the art. The filter (or multiple filters) may be implemented, e.g., as a hardware module within the sensor circuitry 322. Alternatively, the filter may be provided in a software module stored, for example, in the system memory 308.

In more detail, a Kalman filter computes the monitored quantity (such as the pressure in the pump chamber) based on the modeling assumption that the true state of the quantity at time k has evolved from the state at time (k−1) according to

xₖ=Fₖ·xₖ₋₁+Bₖ·uₖ₋₁+wₖ,

where Fₖ is the state-transition model, which is applied to the previous state xₖ₋₁; Bₖ is a control-input model, which is applied to a control vector uₖ; wₖ is the process noise, which is assumed to be drawn from a zero-mean multivariate normal distribution with covariance Qₖ (i.e., wₖ˜N(0,Qₖ)). At time k, an observation (or measurement) zₖ of the true state xₖ is made according to

zₖ=H·xₖ+vₖ,

where Hₖ is the observation model, which maps the true-state space into the observed space, and vₖ is the observation noise, which is assumed to be zero-mean Gaussian white noise with covariance Rₖ (i.e., vₖ˜N(0, Rₖ)). The initial state and the noise vectors at each step {x₀, w₁, . . . , wₖ, v₁ . . . vₖ} are all assumed to be mutually independent. Many real dynamical systems do not exactly fit this model. In fact, unmodeled dynamics can seriously degrade the filter performance, even when it is supposed to work with unknown stochastic signals as inputs, because the effect of unmodeled dynamics depends on the input, and can, therefore, cause instability of the estimation algorithm (i.e., divergence). On the other hand, independent white-noise signals do not make the algorithm diverge. Separating between measurement noise and unmodeled dynamics is a problem addressed by control theory under the framework of robust control (as known to persons of skill in the art).

It is important that a drug pump device (or other automatic liquid-dispensing device) be able to detect if the fluid path is occluded or if delivery of the reservoir contents is complete (i.e., an “end-of-dose” event has occurred for a drug pump designed for one-time use). For traditional motor-driven infusion systems, end-of-dose and occlusion events can be detected simply by monitoring the motor-gear system displacement. Such displacement monitoring for a gear system is, however, not applicable to electrolysis pump systems, which do not have a gear mechanism. Alternative techniques for detecting end-of-dose and occlusion events are therefore needed. Various embodiments of the present invention exploit the fact that, for an electrolysis engine (or other gas-generation engine), the pressure in the pump chamber exhibits a characteristic “pressure signature” when an occlusion or end-of-dose event occurs. Accordingly, monitoring the drug pump device for end-of-dose and/or occlusion events in accordance with various embodiments involves continuously measuring the pressure in the pump chamber, and monitoring the measured gas pressure for end-of-dose and/or occlusion signatures. When one of these types of events is detected, the system controller may initiate an appropriate response, such as shutting down the pump (by interrupting the drive current supplied to the electrolysis electrodes) to avoid over-pressurization of the pump chamber (which would potentially constitute a safety hazard). Alternatively or additionally, further processing of the detected signatures may be performed to discriminate between the two types of events.

In various embodiments, an occlusion or end-of-dose event is detected based on a pressure increase above a certain threshold and/or a significant change in the slope of pressure as a function of time (dp/dt). FIG. 4D illustrates, for example, the pump-chamber pressure throughout delivery of a dose and for a certain time period following delivery. As can be seen, the pump pressure is approximately constant (i.e., it fluctuates by less than 10% of its absolute value) throughout the majority of the delivery (i.e., from a few minutes after start of deliver until the end of delivery at about 12.5 minutes). However, shortly after the volume of liquid in the reservoir has been fully delivered—i.e., after the plunger has completely traversed the vial and hit the vial's front end—the pressure starts rising continuously; this is a consequence of continued gas generation in the pump chamber without any opportunity for volume expansion. A similar pressure increase may be observed if the fluid path is fully or partially occluded. Eventually, the pressure will exceed the set threshold, triggering the declaration of an end-of-dose event or occlusion event. The pressure threshold may be set based on typical pressure fluctuations during normal operation and, optionally, the speed with which the pressure can be expected to rise in the event that the plunger can move no farther (which depends, in turn, on the gas-generation rate). For example, if the pressure typically fluctuates within 10% of its average absolute value, a threshold of 20% above the average pressure may be suitable.

To enable the pump to respond appropriately to a sudden pressure increase, it may be advantageous to discriminate between end-of-dose and occlusion events. In the case of the end of dose delivery, it generally suffices to shut down the electrolysis pump, and possibly to withdraw the needle, cannula, or other delivery vehicle from the patient's subcutaneous tissue. In the event of an occlusion, however, it is desirable to inform the patient thereof (in addition to interrupting pump operation for safety reasons) so that the cause of the occlusion can be found and eliminated and drug-delivery can resume. For example, the pump may perform an automatic diagnostic or occlusion-removal sequence, which may include increasing or decreasing the driving pressure and monitoring the response of the system through one or more sensors, such as the pressure sensor located in the electrolysis chamber. In case of either an end-of-dose or an occlusion event, it may be desirable to relieve the pressure within the electrolysis chamber, e.g., by means of recombination or release of gas through a valve.

A number of different methods are available for distinguishing between end-of-dose events and occlusions. In some embodiments, the cumulatively delivered volume is monitored, and when the sudden pressure increase (or other signature) is detected as the cumulative volume reaches the total volume of drug provided in the vial (or is close), the pressure increase is deemed due to the end of dose. Similarly, in some embodiments, an expected dose-completion time is computed (e.g., based on the total volume to be delivered in conjunction with the gas-generation rate), and if the event occurs close to the expected completion time, an end-of-dose event is declared. Both of these methods depend, in general, on the repeatability of drug delivery, i.e., on repeatable dispensing volumes and times. Of course, these methods can result in false negatives for the detection of occlusions, as occlusions can, in principle, occur near the end of dose. However, it is generally highly probable that a pressure increase that occurs close to the completion time and/or volume signifies an end-of-dose event.

A different means of distinguishing between occlusions and the end of dose may be provided by the pressure signature itself. In devices with a fixed overall volume (i.e., sum of pump-chamber and drug-reservoir volumes), the slope of the pressure change with respect to time, dp/dt, is typically less steep for an end-of-dose event than for an occlusion event. This is because the change in pressure dp/dt is inversely proportional to the amount of gas in the pump chamber, which is greater at the end of the dose when the plunger has reached the front-end of the vial. The slope associated with the end of dose may be calibrated in a bench-top test and stored in memory on the pump device (e.g., in the system memory 308, or in memory of the system controller 306). A comparison of the measured pressure change with the calibrated value may then be used to determine whether an end-of-dose event has happened. Furthermore, in devices that utilize an elastomer plunger and, for the reservoir, a rigid cartridge with a tapered shoulder, the concavity of the pressure with respect to time, i.e., the second derivative d²p/dt² is typically less pronounced (i.e., has a lower value) for an end-of-dose event. The concavity of the pressure results, in this case, from compression of the (cylindrical) plunger into an approximately conically shaped space at the shoulder of the cartridge, and may likewise be calibrated. Accordingly, the second time derivative of the measured pressure may be used discriminate between end-of-dose and occlusion events.

If a single method of detection or differentiation is not sufficiently accurate (as may be the case in some applications), the results of multiple methods may be combined to produce a more accurate result. For example, in some embodiments, the results from multiple methods (e.g., analysis of the second time derivative and comparison of the time and/or cumulative volume at which the event occurs with the expected completion time and/or volume) are combined using a point system in which each result is assigned a certain number of points (or a certain weight), depending on the confidence, associated with the respective method, of accurately predicting or differentiating between events. Once the sum of the points passes a pre-set threshold, the overall confidence may be deemed sufficient to make an accurate determination.

### Integrated Needle-Insertion Systems

Drug pump devices in accordance with various embodiments may be used to deliver an individual drug dosage over several minutes, or to inject drug continuously or in multiple boluses over an even longer period of time (e.g., tens of minutes, hours, or even days). At the shorter end of this range, the drug may be delivered subcutaneously directly through a needle piercing the skin. For prolonged delivery, a soft cannula preferably establishes the fluid path between the drug reservoir and the injection site; in this case, a needle may be used to insert the cannula through the skin, but the needle is thereafter retracted. In either case, the entire volume of drug in the reservoir is generally delivered with only a single needle insertion. To ensure injection at the proper subcutaneous location and prevent trauma to the injection site, it is important to avoid movement of the needle and/or cannula relative to the patient as much as possible. In some embodiments, this is achieved by integrating the needle and/or cannula into the drug pump device and placing the device securely against the patient's skin, typically with a relatively large flat or suitably curved (e.g., to conform to the patient's anatomy) surface, hereinafter the “bottom surface,” of the device housing. In certain embodiments, the drug pump device is attached to the skin via an adhesive skin patch, obviating the need to hold the device against the skin by hand and rendering it wearable. The skin patch may be adhered to the bottom surface of the device, or integrated with the device, e.g., so as to form an envelope around the other device components.

The needle may be inserted through the skin by an automatic, albeit typically manually triggered, mechanism. In certain embodiments, this mechanism, as well as the needle and/or cannula themselves, are integrated with the drug pump device to facilitate ease of use by reducing the number of system components that need to be handled and allowing the patient, once the device is properly placed against the skin, to insert the needle by the push of a button or some other simple and convenient trigger mechanism. To avoid accidental firing of the needle, the device may include a safety interlock, such as, e.g., a second, separate activation mechanism, or a skin sensor that deactivates the needle-insertion mechanism unless and until contact with the skin is detected. (A skin-sensor-based safety interlock is described, for example, in U.S. patent application Ser. No. 13/960,470, filed on Aug. 6, 2013, the entire disclosure of which is incorporated herein by reference.)

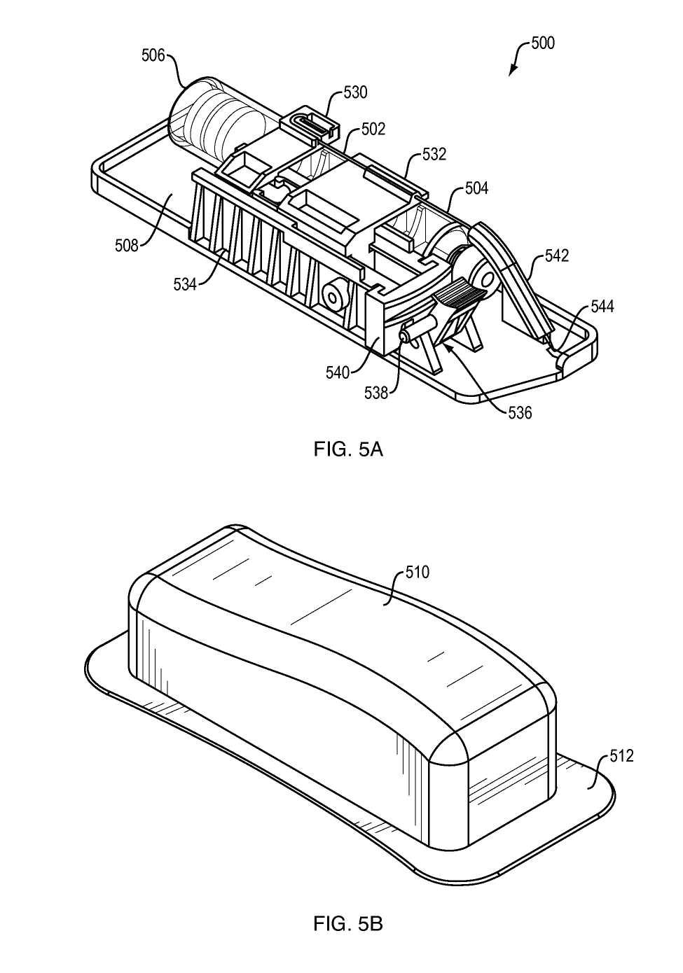

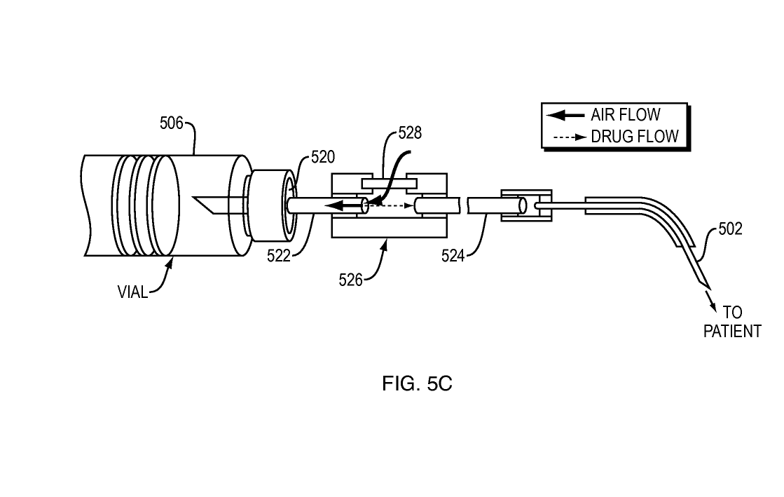

FIG. 5A illustrates an exemplary drug pump device 500 with an integrated insertion mechanism in accordance with various embodiments. The device 500 includes a rigid hollow needle 502 (e.g., made of metal or hard plastic) and, coaxially aligned therewith, a soft cannula 504, typically made of a soft polymer such as, e.g., parylene, polycarbonate, polyvinyl chloride (PVC), or a fluoropolymer (such as polytetrafluoroethylene (PTFE), perfluoroalkoxy (PFA), or fluorinated ethylene propylene (FEP)). The cannula 504 may also be a multi-layered extruded tube made from a combination of multiple of these materials. The needle 502 is utilized to first puncture the patient's skin and to allow the soft cannula 504, which travels along with and is structurally supported and guided by the needle, to penetrate the skin. When the needle 502 and cannula 504 have reached a specified subcutaneous depth, the rigid needle 502 automatically retracts. At first, the needle and cannula travel together, so that the needle punctures the skin and provides structural stability and guidance for the soft cannula. Upon reaching a desired subcutaneous depth, the needle automatically retracts. In the illustrated embodiment, the needle 502 rides within the cannula 504, with a close fit, but not so tight as to prevent the necessary relative sliding. In alternative embodiments, the cannula may, instead, ride within the hollow needle. Either way, the hollow needle 502 may be sealed in relation to the soft cannula, and form part of the fluid path, even after needle retraction.

As illustrated, the needle 502 and cannula 504 ride above, with their axes parallel to, the drug vial 506. The drug vial 506, in turn, is placed in the device parallel to the bottom wall 508 of the housing. The outer surface of the bottom wall 508 is designed for placement against the patient's skin; thus, the vial 506 and portions of the needle 502 and cannula 504 are, during use, oriented parallel to the skin. Positioning the needle 502 and/or cannula 504 above or, in alternative embodiments, below the vial 506, as opposed to side-by-side with the vial, may have several advantages. For example, it allows the flexible tubing connecting the drug vial 506 to the needle 502 (as described further below) to be routed on the opposing side of the vial 506, preventing entanglement with the components of the insertion mechanism (such as the needle and cannula carriers, track, and springs, also described further below). Further, it can improve human factors by aligning the needle 502 with the vial 506, thereby providing cues that make the location of the injection site relative to the device more intuitive. It may also allow the needle 502 to be visualized through the same window in the device housing as the vial 506. In addition, needle/cannula positioning above or below the vial 506 may improve ease of assembly by moving the fluid path away from the insertion mechanism, and may provide improved access of assembly equipment for easier welding of components required for the needle-cannula interface seal. In FIG. 5A, only the bottom wall 508 of the device housing is shown to reveal the interior components of the device for illustration purposes. Typically, however, all device components are fully enclosed with a housing 510, as shown in FIG. 5B. The device 500 may be attached to the skin with an adhesive patch 512 placed between the bottom surface 508 and the skin.

To deploy the device 500 for drug delivery, a fluid path needs to be established between the drug vial 506 and the injection site. As shown in FIG. 5C, establishing the fluid path involves, in various embodiments, (i) piercing the septum 520 at the vial outlet (e.g., septum 104 shown in FIG. 1) with a hollow, rigid septum-puncturing needle 522 (e.g., made of metal or hard plastic) or a molded plastic spike, which is fluidically connected to the needle 502 intended to drive the cannula 504 through the skin, and (ii) inserting the needle 502 and cannula 504 into the subcutaneous tissue. The septum needle 522 may sit within the housing, and may be covered with an elastomer seal or other seal that provides a sterile barrier for the needle 522, but can be punctured. Alternatively, the sterile barrier may be mechanically removed from the needle 522 prior to establishing the fluid path. In yet another embodiment, the entire device 500 is maintained within sterile packaging until deployed by the user.

The septum needle 522 may be connected via flexible tubing 524 (made, e.g., of parylene, phthalate-free (in particular, Di(2-ethylhexyl)phthalate-(DEHP)-free) PVC, ethylene-vinyl acetate (EVA), low-density polyethylene (LDPE), polyurethane, tygone, silicone, or another soft polymer) to the injection needle 502 and cannula 504. The tubing may also be multi-layered, and may have a surface coating for drug compatibility. The connection between the septum needle 522 and the flexible tubing 524, in turn, may be established by a fluid-path connector 526 that forms a channel section into whose ends the needle 522 and tubing 524 can be seal-fitted. The fluid-path connector 526 may be contained within or be part of a septum-needle hub (not shown), which may also provide structural support for securing the vial 506 in the pump housing (including, e.g., during device manufacture while a force is applied to the vial to seal it against the electrolysis chamber). In general, the hub may be fixed or moveable. For example, the hub may be moveable through user action, such as pushing of a button on the external envelope of the device, which may correspond directly to an internal movement (e.g., a sliding action) of the hub or indirectly cause movement of the hub, e.g., via release of a spring; hub movement may result in the puncturing of the vial septum 520 with the septum needle 522. Alternatively, if the hub is fixed, the housing may be movable.

The fluid-path connector 526 and/or hub may include a vent or filter 528 that allows air, but not liquid, to pass through; such a vent/filter may be made, for example, of a fluoropolymer (e.g., of PTFE or polyvinylidene fluoride (PVDF)) membrane or of unsaturated polyester (UPE) of a mofied acrylic. The vent or filter 528 may serve two purposes: On the one hand, it may allow air bubbles that have been trapped in the drug vial 506 to escape through the vent/filter 528, preventing the injection of air into the patient. On the other hand, under circumstances in which a below-atmospheric pressure or vacuum pressure is created in the drug vial 506, the vent/filter 528 may allow air to fill the void, preventing suction in the fluid path from pulling bodily fluids or tissue from the injection site (e.g., blood and/or drug) into the device. Low pressure in the drug vial 506 can result, for instance, from the recombination of electrolysis gases in the pump chamber; such recombination may be induced deliberately (e.g., by introduction of a catalyst or via spark- or heat-ignited combustion) upon emptying of the drug vial 506 to allow for the safe handling and/or disposal of the pump device. However, recombination may cause a vacuum in the pump chamber which, via retraction of the plunger, may create suction in the vial 506 and can, if the pump device is still in fluid communication with the patient's body, draw matter from the site where liquid drug was just dispensed. This undesirable effect is undermined by the vent or filter 528.

In some applications, the fluid path downstream of the drug vial 506 (or a segment of the fluid path) needs to be primed prior to infusion, i.e., liquid drug needs to be pumped into the fluid path before a fluid connection with the injection site in the patient is established. This can be achieved by briefly operating the electrolysis pump following insertion of the septum needle 522 through the vial septum 520. Alternatively, priming may be accomplished by a separate, e.g., manual, mechanism. For example, the user may actuate a button, lever, or other mechanical element on the housing to move the plunger slightly to dispense the liquid drug.

With renewed reference to FIG. 5A, to facilitate automatic insertion of the needle 502 and cannula 504 into the subcutaneous tissue upon actuation of a suitable trigger, the pump device 500 has an integrated insertion mechanism. This insertion mechanism includes a needle carrier 530 and a cannula carrier 532 slidably mounted within a rail 534, which is, in the depicted embodiment, arranged side-by-side with and parallel to the drug vial 506. The needle 502 and cannula 504 are fixedly mounted in the needle carrier 530 and cannula carrier 532, respectively, such that linear movement of the carriers 530, 532 within the rail 534 translates directly into movement of the needle 502 and cannula 504 along their axes. Mechanical compression springs (not shown) disposed inside the rail 534 may be used to cause motion of the carriers 530, 532 when actuated by the user. For example, as explained in more detail below, when the user actuates a rotating lever 536 while the safety interlock 538 allows actuation, an insertion spring may be released, driving the needle and cannula carriers 530, 532 forward to thereby advance the needle 502 and cannula 504. Once the cannula carrier 532 hits the end stop 540 of the rail 534, this forward motion comes to a halt, the needle and cannula carriers 530, 532 are decoupled, and a refraction spring is released to push the needle carrier 532 backward, thereby withdrawing the needle 502. The geometries of and spatial clearances between the sliding components are generally designed such that no binding, sticking, or over-constraining takes place during this process.

The pump device 500 may include a needle guide 542 that, upon advancement of the injection needle 502 and cannula 504, deflects the needle 502 and cannula 504 from their horizontal axis (i.e., the axis parallel to the bottom wall 508) and guides them downward at a pre-determined angle toward an opening 544 in the bottom wall 508 of the device housing (which, if an adhesive patch is used, is generally aligned with an opening in the patch). Significant needle bending is possible, despite the rigid material from which the needle 502 is typically made, if the needle wall is sufficiently thin (e.g., between 0.002 and 0.006 inches). The needle guide 542 may define a curved guide channel aligned, at one end, with the axis of the needle 502 and, at the other end, with the opening 544, and may be formed integrally with the housing or a part thereof (e.g., with the bottom wall 508 or the device cover (not shown)), or provided as a separate component secured to the housing or other part of the device. In some embodiments, the needle guide 542 also provides support for and/or aids in the alignment of the septum needle 522; for example, the septum-needle hub may be integrated with the needle guide.

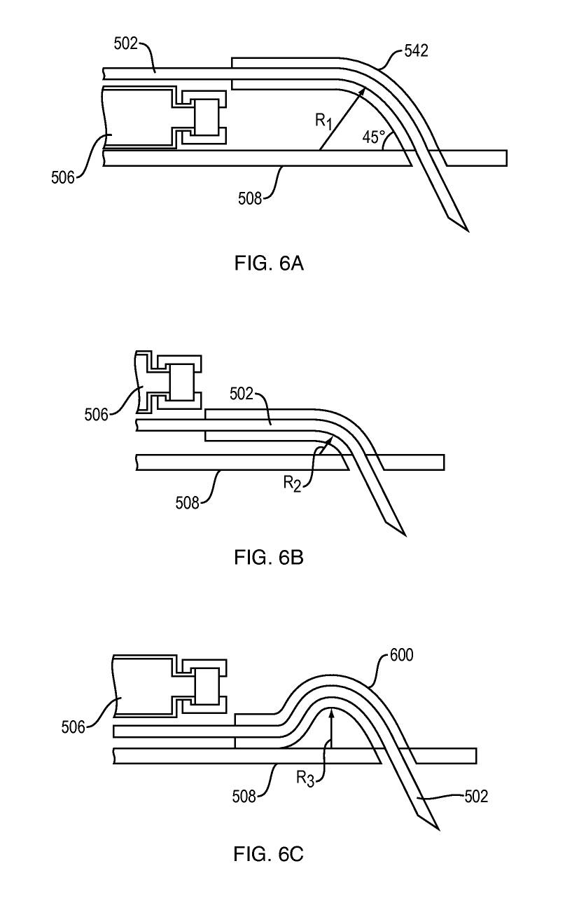

For many subcutaneous drug-delivery applications, it is desirable to insert the needle 502 and/or soft cannula 504 into the patient's skin at a 45° angle, which typically helps minimize pain and discomfort during the injection. However, other angles (e.g., 30° or 90°) are possible and may, in some circumstances, be desirable. The needle guide 542 can generally be designed to direct the needle 502 and/or cannula 504 into the skin at the desired angle. In various embodiments, as shown in FIGS. 5A and 6A, the needle 502 and cannula 504 pass over the drug vial 506 (as shown in FIG. 5A), allowing for a generous bend radius (e.g., radius R₁ in FIG. 6A) that prevents kinking of and damage to the (relatively rigid) needle 502. In other embodiments, shown in FIG. 6B, the needle 502 and cannula 504 pass under the drug vial 506 (i.e., closer to the bottom wall 508). Advantageously, this may shorten the fluid path between the vial 506 and the needle 502, allow use of a shorter needle 502, bring the needle 502 closer to the patient's skin, and/or facilitate visualization of the drug vial 506 from above (as the needle 502 does not occlude the view of the vial 506). Passing the needle 502 and/or cannula 504 under the vial 502 allows, however, only for a small bend radius (e.g., radius R₂). This may be acceptable if a shallow puncture and penetration angle (e.g., an angle of 30° or less) is desired. For larger angles, however, the small bend radius can lead to damage to the needle 502. This problem can be overcome with a needle guide 600, shown in FIG. 6C, that directs the needle 502 and cannula 504 upwards before directing them downwards into the patients skin, creating an increased bend radius R₃ that prevents damage to the needle 502 (while allowing the needle 502 and cannula 504 to pass under the vial).

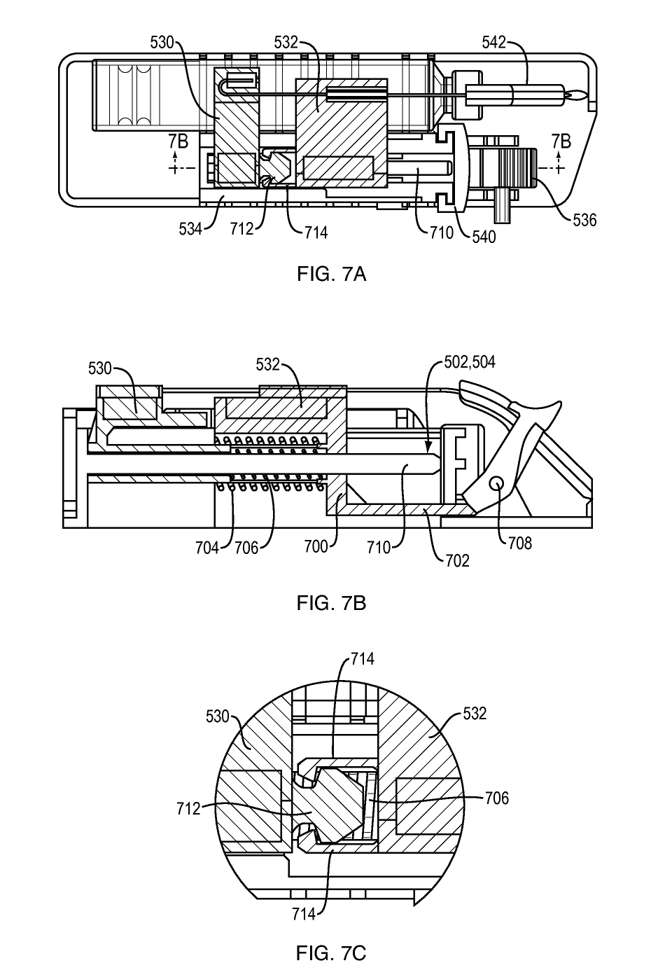

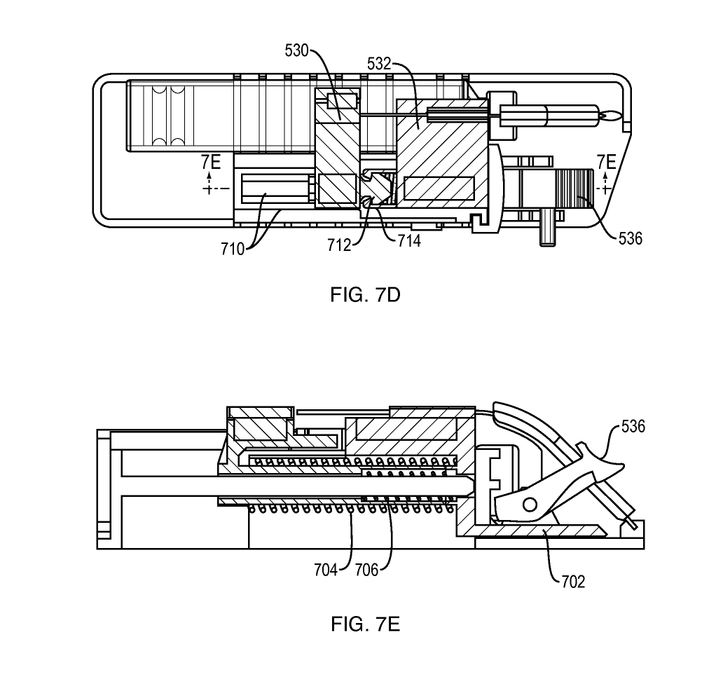

The needle insertion mechanism of the pump device 500 is illustrated in more detail in FIGS. 7A-7E. FIGS. 7A and 7B show top and side views of the device 500 prior to actuation. In this state, both carriers 530, 532 are retracted; therefore, neither the needle 520 nor the cannula 504 extends outside the envelope of the device 500. The cannula carrier 532 includes a front wall 700 extending farther down into the rail 534 than the main carrier body and, extending from the lower end of this front wall 700, a tongue 702 sized to fit through a corresponding slot in the end stop 540. The slot is blocked, however, by the lever 536 prior to actuation. Thus, advancement of the cannula carrier 532 is prevented, as is advancement of the needle carrier 530, which is located behind the cannula carrier 532. The insertion spring 704 is compressed and, due to its placement between the front wall 700 of the cannula carrier 532 and a recess in the rail 534, prevented from expanding. The retraction spring 706 is nested inside the insertion spring, between the cannula-carrier front wall 700 and a front end of the needle carrier 530, and is likewise in the compressed state.

The lever 536 pivots about a horizontal axis 708 that is well-supported by a structural element of the housing or a scaffold therein. Upon rotation of the lever 536 around this axis, the slot in the end stop 540 is unblocked, releasing the cannula carrier 532. The cannula carrier 532 advances, urged forward by the insertion spring 704. At the same time, the needle carrier 530 advances, pulled by the cannula carrier 532, e.g., via a latch mechanism. The carriers 530, 532 may travel along a guiding rod 710 that is support only on one end to prevent binding (i.e., over-constraining) The latch mechanism, shown in a close-up view in FIG. 7C, may include a latch head 712 attached to or forming an integral part of the needle carrier 530 and, locked around the head 712, two latches 714 attached to or forming an integral part of the cannula carrier 532. The latches 714 are designed and sized so that they cannot inadvertently release the needle carrier 530. The retraction spring travels along between the needle and cannula carriers 530, 532, without affecting the expansion rate or driving force of the insertion spring. Although the compressed retraction spring is located to exert forces that push the needle carrier 530 away from the cannula carrier 532, separation between the carriers 530, 532 is prevented by the latch mechanism. Since both the needle and cannula carriers 530, 532 travel together, the needle 502 and cannula 504 travel together as well. In this configuration, the needle 502 and cannula 504 are guided downward, by the needle guide 542, toward the skin (e.g., at a 45° angle).

The advancing motion ceases once the cannula carrier 532 reaches its physical end of travel when hitting the end stop 540; FIGS. 7D and 7E illustrate the device in this state. The device is configured such that, at this point, both the needle 502 and cannula 504 have punctured and penetrated the skin to a desired depth. Further, when or shortly before the cannula carrier 532 reaches the end stop 540, the latch mechanism releases the needle carrier 530: As shown in FIG. 7C, the latches 714 include thin supporting segments that can deflect outward in the absence of any constraint. Prior to actuation and thereafter during the initial advancement of the needle and cannula carriers 530, 532, the latches 714 are constrained by the adjacent side walls of the rail 534 or housing, and can therefore not deflect. As the cannula carrier 532 reaches its end of travel, however, the wall(s) that keep the latches from deflecting recede, no longer preventing the latches 714 from bending outward. Due to their contact angle and the offset force, the latches 714 deflect outwardly, away from the needle carrier 530; the contact angle is selected to ensure release at the right time, while preventing jams or self-locking of the mechanism. The needle carrier 530 is then free to move, and slides away from the cannula carrier 532 due to the action of the retraction spring. This action, in turn, retracts the needle 502, leaving only the cannula 504 under the skin.

Of course, the device 500 described with respect to FIGS. 5A-7E is only one exemplary embodiment, and many modifications and variations will be readily apparent to one of skill in the art. For example, while the stop member that prevents cannula-carrier advancement prior to actuation is, in the illustrated embodiment, an integral part of the lever 536 (which, advantageously, reduces part count), this need not be the case. Instead, the stop member and lever—or other component or mechanism for triggering the insertion mechanism—may be separate devices; in such arrangements, the triggering component does not bear the spring forces directly. Further, the triggering component need not be a lever, but may be, e.g., a push-button, pull-button, sliding button, rotating component, or any other suitable element or feature. In addition, the triggering mechanism may include two or more buttons or equivalent triggering components, e.g., two buttons, each located on opposite sides of the device. The triggering component or mechanism may also perform additional functions, such as activating the electronics, switching the power on, initiating the pumping sequence, etc. Further, a safety mechanism, e.g., a safety interlocking component or programmed procedure, may be employed to prevent accidental actuation of the device. In some embodiments, the safety mechanism is conveniently activated and disabled by user manipulation, i.e., without requiring additional internal actuators, such as motors, solenoids, etc.

In the illustrated configuration, the latches 714 are integrated with the cannula carrier 532 and symmetrically placed around the latch head 708. In other embodiments, however, the latches 714 may instead be located on the needle carrier 530 or another component. Further, the latches 714 need not be symmetrical about a cross-sectional plane, and may be oriented differently from the manner illustrated (e.g., at an angle or vertically, in relation to the axis of carrier travel). Alternatively or in addition to being constrained by side walls, the latches 714 may be constrained from the top and/or bottom. The latches 714 may also be mechanically assisted to release the needle carrier 530 (at the appropriate time) by physically contacting other elements. The physical ends of travel may be cushioned by, e.g., an elastomer, shock absorber, integral rigid spring, etc. to reduce device shock and sound level.

The insertion mechanism may be designed to permit assembly of the carriers, springs, and other components from one end of the housing, providing for simple assembly that may be done manually, semi-automatically, or automatically. The carriers and springs may form a subassembly that is put together separately and then introduced into the device housing. The cannula and/or needle may be guided at different angles with respect to the insertion/retraction mechanism and may follow different paths, according to the design preferences. The cannula/needle may also be offset from the points at which they are attached to the carriers, and guided to protrude from the device at a desired location (e.g., centered in relation to the body of the device).

The terms and expressions employed herein are used as terms and expressions of description and not of limitation, and there is no intention, in the use of such terms and expressions, of excluding any equivalents of the features shown and described or portions thereof. Rather, having described certain embodiments of the invention, it will be apparent to those of ordinary skill in the art that other embodiments incorporating the concepts disclosed herein may be used without departing from the spirit and scope of the invention. Further, embodiments of the invention may possess any or all of the features and advantages described herein, in any suitable combination, even if such combinations were not made express herein. For example, while the embodiments described above generally refer to electrolytically driven drug pump devices, certain inventive aspects, such as needle-insertion mechanisms integrated into drug pump devices, may be applicable to devices with other pump mechanisms as well. Conversely, the above-described features of electrolysis pumps may be used in drug-delivery devices with or without integrated needle-insertion mechanisms. Accordingly, the described embodiments are to be considered in all respects as only illustrative and not restrictive.

What is claimed is:

1. A drug pump device comprising:
a housing comprising a bottom wall having an outer surface for placement in contact with a patient's skin;
contained within the housing, (i) a drug vial having an outlet at a first end thereof and forming a drug reservoir therein, (ii) a plunger movably disposed within the vial, and (iii) a pump attached to the drug vial at a second end thereof for causing movement of the plunger so as to expel drug from the outlet of the vial, the vial being oriented parallel to the bottom surface;
disposed within the housing either above or below the vial and parallel thereto, a needle fluidically connectable to the outlet of the drug vial;
disposed within the housing, an insertion mechanism for advancing the needle along an axis thereof; and
disposed within the housing on the bottom wall, a curved needle guide, aligned, at an upper end thereof, with the axis of the needle, for bending the needle upon advancement thereof so as to guide a tip of the needle toward and through a plane coinciding with the bottom wall.

2. The drug pump device of claim 1, wherein the insertion mechanism comprises a needle carrier slidably movable relative to the housing in a direction parallel to the needle axis.

3. The drug pump device of claim 2, wherein the insertion mechanism further comprises an insertion spring for advancing the needle so as to advance the needle through the plane coinciding with the bottom wall of the housing.

4. The drug pump device of claim 3, wherein the insertion spring is placed off-axis from the needle and side-by-side with the vial.

5. The drug pump device of claim 4, wherein the insertion mechanism further comprises a retraction spring for retracting the needle carrier whereby the needle is retracted into the housing.

6. The drug pump device of claim 5, wherein the retraction spring is disposed off-axis from the needle and concentrically with the insertion spring.

7. The drug pump device of claim 4, further comprising a cannula disposed concentrically with the needle and fluidically connectable thereto.

8. The drug pump device of claim 7, wherein the needle is configured to ride within the cannula.

9. The drug pump device of claim 7, wherein the needle is hollow and the cannula is slidably received therein.

10. The drug pump device of claim 7, wherein the insertion mechanism further comprises a cannula carrier slidably movable relative to the housing in a direction parallel to the cannula axis.

11. The drug pump device of claim 10, wherein the insertion mechanism further comprises a latch-and-release structure for (i) causing the needle and cannula carriers to move together during advancement and (ii) upon insertion of the needle and cannula through the bottom surface of the housing, detaching the cannula carrier from the needle carrier to allow movement of the needle carrier relative to the cannula carrier.

12. The drug pump device of claim 1, further comprising a manually operable trigger mechanism for triggering the insertion mechanism.

13. The drug pump device of claim 1, wherein the bottom wall comprises an opening, the needle guide being aligned, at a lower end thereof, with the opening so as to guide the needle tip therethrough.

14. The drug pump device of claim 1, further comprising a skin-adhesive patch attached to the outer surface of the bottom wall.

15. The drug pump device of claim 1, further comprising a fluid-path connector for connecting the outlet of the drug vial to the needle, the fluid-path connector comprising a vent permeable to air but not liquid.

16. The drug pump device of claim 1, wherein the needle is disposed below the vial and the needle guide is curved to guide the needle first away from the bottom wall and then back toward the plane coinciding with the bottom wall.

17. A drug pump device comprising:
a housing and, contained therein, a drug vial having an outlet at a first end thereof and forming a drug reservoir therein, a plunger movably disposed within the vial, and a pump attached to the drug vial at a second end thereof for causing movement of the plunger so as to expel drug from the outlet of the vial, the vial being oriented parallel to the bottom surface;
disposed within the housing, a needle fluidically connectable to the outlet of the drug vial; and
a fluid-path for connecting the outlet of the drug vial to the needle, the fluid-path connector comprising a vent permeable to air but not liquid.

18. A drug pump device comprising:
a housing comprising a bottom wall having an outer surface for placement in contact with a patient's skin;
contained within the housing, a drug vial having an outlet at a first end thereof and forming a drug reservoir therein, a plunger movably disposed within the vial, and a pump attached to the drug vial at a second end thereof for causing movement of the plunger so as to expel drug from the outlet of the vial, the vial being oriented parallel to the bottom surface;
disposed within the housing below the vial and parallel thereto, a needle fluidically connectable to the outlet of the drug vial; and
disposed within the housing on the bottom wall, a curved needle guide, aligned, at an upper end thereof, with the axis of the needle, for bending the needle upon advancement thereof so as to guide a tip of the needle first away from the bottom wall and then back toward an opening in the bottom wall.
